(19) United States
(12) Patent Application Publication
(10) Pub. No.: US 2011/0028964 A1
(43) Pub. Date: Feb. 3, 2011

(54) ELECTROSURGICAL INSTRUMENT

(76) Inventor: Thomas R. K. EDWARDS, Salisbury (GB)

Correspondence Address: NIXON & VANDERHYE, PC, 901 NORTH GLEBE ROAD, 11TH FLOOR, ARLINGTON, VA, 22203, US

(21) Appl. No.: 12/842,491

(22) Filed: Jul. 23, 2010

Related U.S. Application Data
(60) Provisional application No. 61/231,332, filed on Aug. 5, 2009.

(30) Foreign Application Priority Data
Jul. 28, 2009 (GB) 0913120.2

(51) Int. Cl. A61B 18/14; A61B 18/18

(52) U.S. Cl. 606/33; 606/48

(57) ABSTRACT

A bipolar electrosurgical instrument (12) is provided for cutting and sealing tissue, the instrument comprising first and second jaw members (4 and 5). The first jaw member (4) has an inner surface (7) adapted to cooperate with the inner surface (21) of the second jaw member (5) for grasping tissue therebetween. A first, coagulating electrode (13) is provided on the inner surface of one of the jaw members, and a second, coagulating electrode (15) is present on the inner surface of one of the jaw members, with an insulating member (9) separating the first and second electrodes. A third, cutting electrode (41) is provided on the inner surface of the first jaw member, and a fourth electrode (19) is provided on an outer surface (8; 48) of the first jaw member, separate from the inner surface (7). The electrosurgical instrument (12) is capable of selectively causing coagulation of tissue between the first and second electrodes (13 and 15), and/or the cutting of tissue contacted by the third electrode (41), and/or the treatment of tissue contacted by the fourth electrode (19).

This invention relates to an electrosurgical instrument for cutting and sealing tissue. Such an instrument is commonly used for the cutting and/or coagulation of tissue in surgical intervention, most commonly in “keyhole” or minimally-invasive surgery, but also in “open” surgery.

It is known to provide an electrosurgical instrument in which the cutting of tissue is effected by means of an elongate electrosurgical electrode extending along the inner surface of one of a pair of jaw elements. U.S. Pat. Nos. 6,174,309 and 7,204,835 are two examples of this kind of instrument. The present invention attempts to provide an improvement to an electrosurgical instrument such as this.

Accordingly, an electrosurgical instrument for cutting and sealing tissue is provided, the instrument comprising:

a) first and second jaw members in opposing relation relative to one another, the first jaw member having an inner surface adapted to cooperate with the inner surface of the second jaw member for grasping tissue therebetween, at least one of the jaw members being movable with respect to the other such that the jaw members are selectively operable between an open position in which the jaw members are disposed in spaced relation relative to one another, and a closed position in which the inner surfaces of the jaw members cooperate for grasping tissue therebetween;

b) means for causing movement of the or each jaw member so as to operate the jaw members between the open and closed positions;

c) a first, coagulating electrode on the inner surface of one of the jaw members;

d) a second, coagulating electrode on the inner surface of one of the jaw members;

e) an insulating member separating the first and second electrodes, the first and second electrodes being connectable to opposite poles of an electrosurgical generator;

f) a third, cutting electrode on the inner surface of the first jaw member, the third electrode being connectable to one pole of an electrosurgical generator; and

g) a fourth electrode on an outer surface of the first jaw member, separate from the inner surface, the fourth electrode being connectable to one pole of an electrosurgical generator;

wherein the electrosurgical instrument is capable of selectively causing coagulation of tissue between the first and second electrodes, and/or cutting of tissue contacted by the third electrode, and/or treatment of tissue contacted by the fourth electrode.

The provision of electrodes on both the inner and outer surfaces of the jaw member allows either the treatment of tissue grasped by the jaw members, or the treatment of tissue adjacent to the jaw members. According to a typical arrangement, the fourth electrode is conveniently a cutting electrode capable of cutting tissue contacted thereby. In this way, the instrument provides for the cutting of tissue grasped by the jaw members, or the cutting of tissue adjacent to the jaw members.

According to an alternative arrangement, the fourth electrode is conceivably a coagulating electrode capable of coagulating tissue contacted thereby. This arrangement allows for tissue grasped between the jaw members to be coagulated (by means of the first and second electrodes), and also for tissue adjacent to the jaw members to be coagulated (by means of the fourth electrode). In this way, the surgeon can select whether to grasp the tissue in order to perform coagulation, or whether to press the jaw members against adjacent tissue, in order to perform “spot” coagulation.

Preferably, the fourth electrode is an electrode capable of selectively cutting or coagulating tissue contacted thereby. In this way, the surgeon can choose whether to perform a cutting or coagulating action upon tissue adjacent the jaw members, by means of the fourth electrode.

The first and second electrodes may be provided on the first jaw member, along with the third and fourth electrodes. This enables the second jaw member to be essentially passive, and the second jaw member conveniently has a tissue-contacting surface which is electrically insulating over its entire surface.

The third electrode preferably extends beyond the inner surface of the first jaw member, and it is conveniently resiliently mounted with respect to that jaw member. Similarly, the fourth electrode conveniently extends beyond the outer surface of the first jaw member, and is also conceivably resiliently mounted with respect to that jaw member. Alternatively or additionally, the fourth electrode is conceivably retractably mounted with respect to that jaw member, such that it is movable between a retracted position in which it is below the outer surface of that jaw member, and an exposed position in which it extends beyond the outer surface of the jaw member. In this way, when the surgeon is not using the fourth electrode, it can be retracted so as not to inadvertently contact tissue adjacent to the jaw members. Alternatively, a removable cover is conveniently provided for selectively covering and uncovering the fourth electrode. When not in use, the fourth electrode can be covered in this way so as not to inadvertently contact tissue.

In one convenient arrangement, the third and fourth electrodes are separate electrodes. They are preferably capable of independent energisation by the electrosurgical generator, so that the surgeon can choose to energise either the third electrode or the fourth electrode (or both). In one convenient arrangement, the third and fourth electrodes are connected one to the other via a capacitor. In this way, both the third and fourth electrodes can be connected to the same pole of the electrosurgical generator, and can be energised at the same time, and yet a different tissue effect can be obtained from the third and fourth electrodes. For example, a more aggressive cut can be obtained from the third electrode as compared to the fourth electrode, or vice versa.

Alternatively, the third and fourth electrodes are integrally formed to constitute a single electrode. While this does not allow for independent or differential energisation, this does provide a simple and effective unitary construction.

The fourth electrode can be positioned on the rear face of the first jaw member, opposite the inner surface thereof. Alternatively, the fourth electrode can be positioned on a side face of the first jaw member, so as to allow for the instrument to be used in a sideways manner. Whichever way in which the fourth electrode is mounted, it provides the surgeon with more options for tissue treatment, without needing to re-orient the jaw members.

According to a further aspect of the invention there is provided an electrosurgical system for cutting and sealing tissue, the system comprising an electrosurgical generator and a bipolar electrosurgical instrument, the instrument comprising:

a) first and second jaw members in opposing relation relative to one another, the first jaw member having an inner surface adapted to cooperate with the inner surface of the second jaw member for grasping tissue therebetween, at least one of the jaw members being movable with respect to the other such that the jaw members are selectively operable between an open position in which the jaw members are disposed in spaced relation relative to one another, and a closed position in which the inner surface of the jaw members cooperate for grasping tissue therebetween;

b) means for causing movement of the or each jaw member so as to operate the jaw members between the open and closed positions;

c) a first, coagulating electrode on the inner surface of one of the jaw members;

d) a second, coagulating electrode on the inner surface of one of the jaw members;

e) an electrosurgical generator for supplying RF energy to the electrosurgical instrument;

f) an insulating member separating the first and second electrodes, the first and second electrodes being connected to opposite poles of the electrosurgical generator;

g) a third, cutting electrode on the inner surface of the first jaw member, the third electrode also being connected to one pole of the electrosurgical generator; and

h) a fourth electrode on an outer surface of the first jaw member, separate from the inner surface, the fourth electrode also being connected to one pole of the electrosurgical generator;

the electrosurgical generator being such as to supply RF energy such that the electrosurgical instrument causes coagulation of tissue between the first and second electrodes, and/or cutting of tissue contacted by the third electrode, and/or treatment of tissue contacted by the fourth electrode.

As described previously, the electrosurgical generator conveniently supplies cutting RF energy to the fourth electrode such that the electrosurgical instrument causes cutting of tissue contacted by the fourth electrode. Alternatively, the electrosurgical generator supplies coagulating RF energy to the fourth electrode such that the electrosurgical instrument causes coagulation of tissue contacted by the fourth electrode, or conceivably the electrosurgical generator selectively supplies cutting RF energy and coagulating RF energy to the fourth electrode such that the electrosurgical instrument selectively causes tissue contacted by the fourth electrode to be either cut or coagulated respectively.

The invention will now be further described, by way of example, with reference to the drawings, in which:

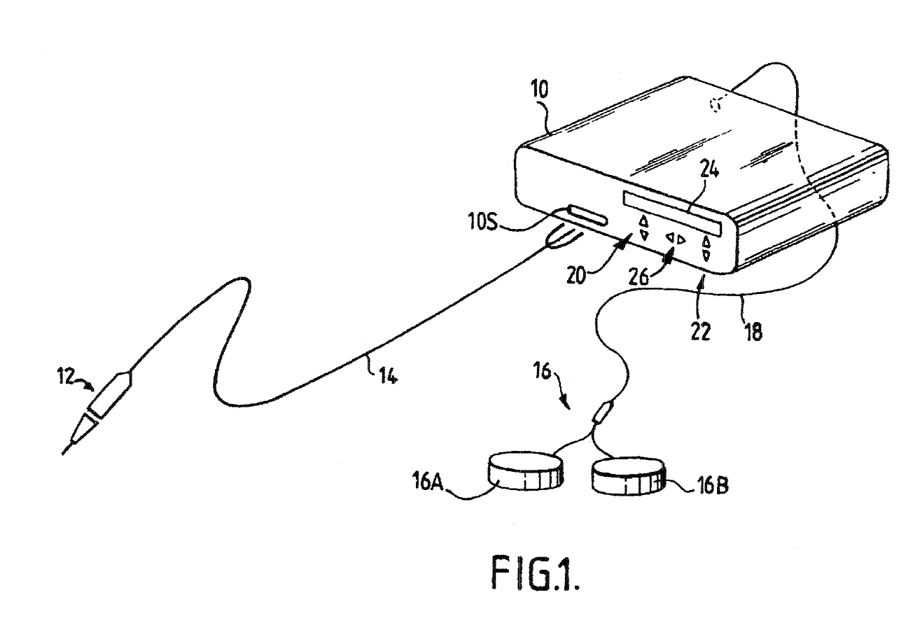

FIG. 1 is a schematic diagram of an electrosurgical system constructed in accordance with the present invention,

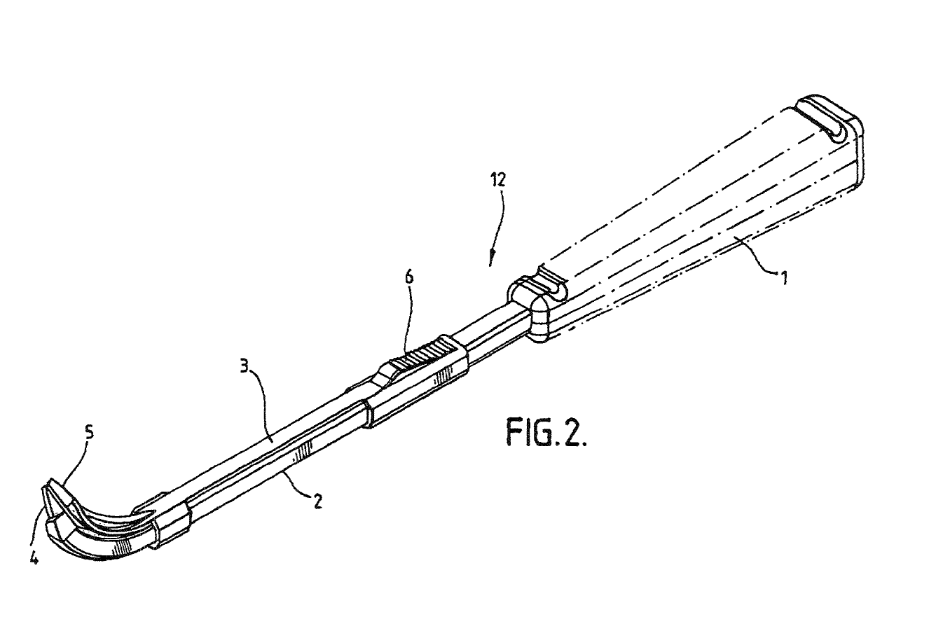

FIG. 2 is a schematic plan view of an electrosurgical instrument constructed in accordance with the present invention and capable of being used in the system of FIG. 1,

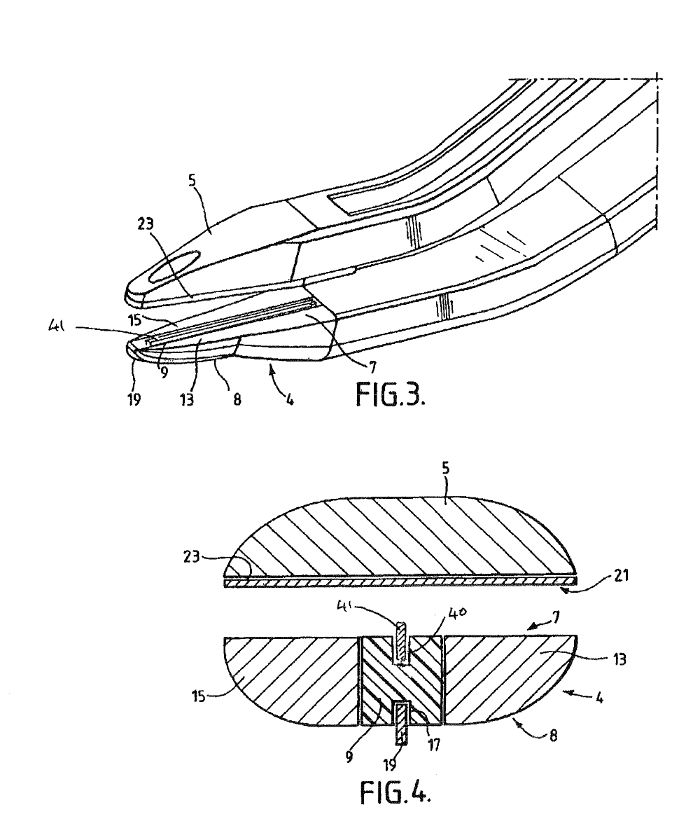

FIG. 3 is a perspective view of the distal part of the electrosurgical instrument FIG. 2,

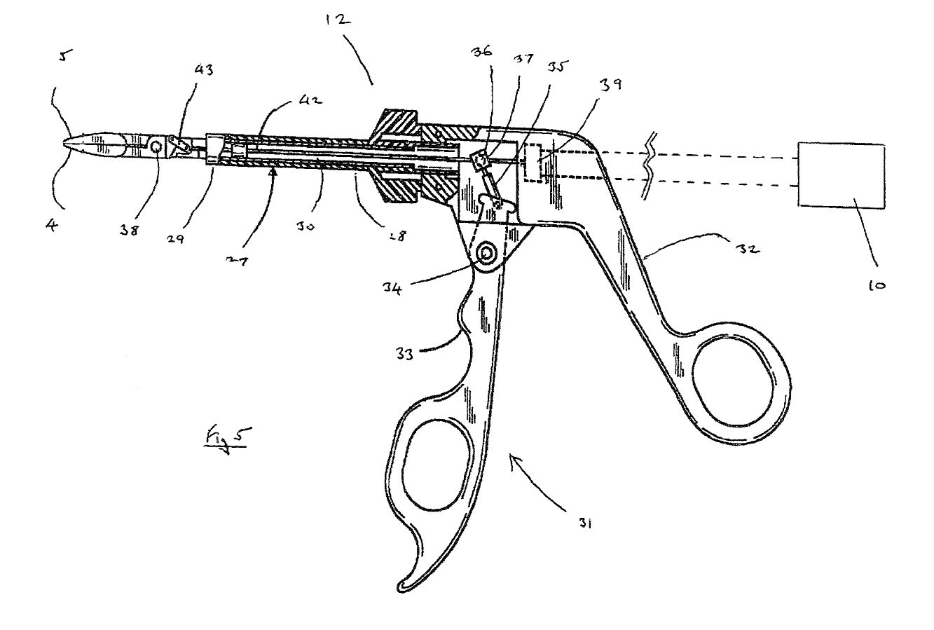

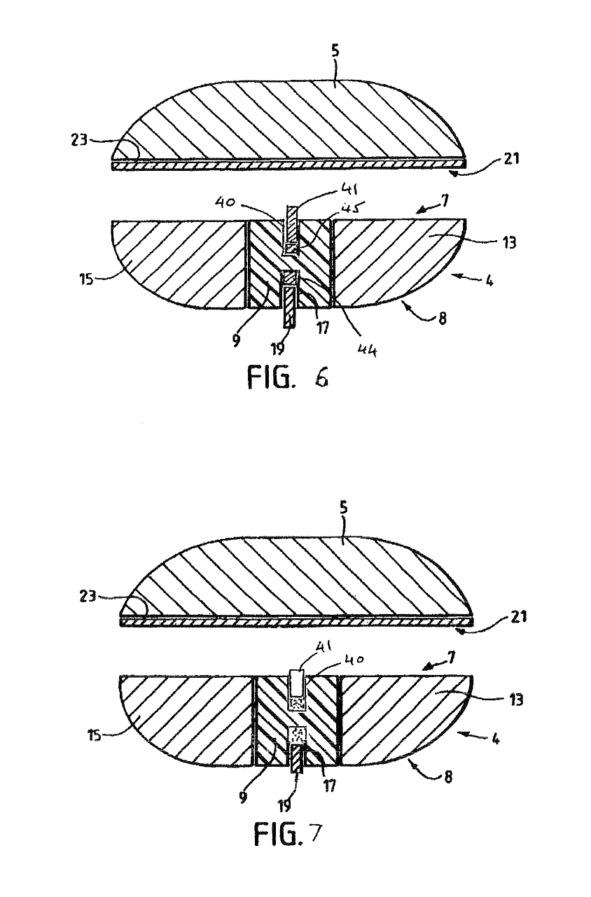

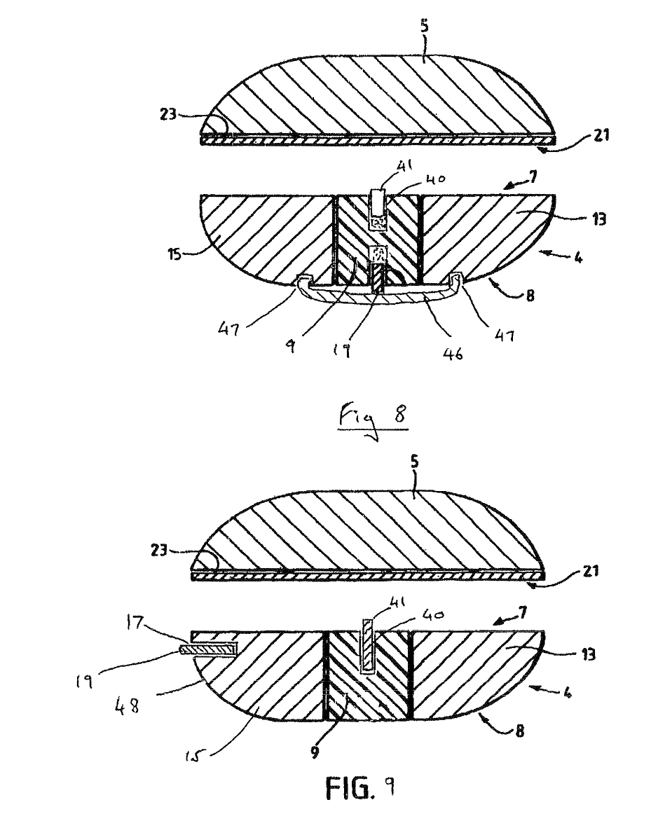

FIG. 4 is a schematic cross-sectional view through the jaws of the instrument of FIG. 3,

FIG. 5 is a schematic view, shown partly in cross-section, of an alternative embodiment of electrosurgical instrument used in the system of FIG. 1,

FIGS. 6 to 10 are schematic cross-sectional views through the jaws of alternative embodiments of an instrument constructed in accordance with the present invention, and

FIGS. 11A & 11B are schematic diagrams of a switching circuit for use with a generator forming part of the system of FIG. 1.

Referring to FIG. 1, an electrosurgical generator 10 has an output socket 10S providing a radio frequency (RF) output for an instrument 12 via a connection cord 14. Activation of the generator 10 may be performed from the instrument 12 via a connection in the cord 14, or by means of a footswitch unit 16, connected to the rear of the generator by a footswitch connection cord 18. The footswitch unit 16 has two footswitches 16A and 16B for selecting a coagulation mode and a cutting mode of the generator 10 respectively. The front panel of the generator 10 has push buttons 20 and 22 for respectively setting coagulation and cutting power levels, which are indicated in a display 24. Push buttons 26 are provided as a means for selection between alternative coagulation and cutting waveforms.

FIG. 2 shows one arrangement of the instrument 12, which comprises a handle 1 from which depends a first arm 2 and a second arm 3 attached thereto. The first arm 2 terminates in a first jaw member 4, while the second arm 3 terminates in a second jaw member 5. The second arm 3 is removably clipped over the first arm 2, and is longitudinally slidable there along. Longitudinal movement of the second arm 3, by means of a finger button 6, varies the distance between the first and second jaw members 4 and 5.

Referring to FIGS. 3 and 4, the first jaw member 4 has a substantially planar inner surface 7, and a curved outer surface 8. The jaw member 4 comprises a central block 9 of insulating material, flanked on either side by first and second electrode 13 and 15, formed of an electrically-conductive material such as copper. The electrodes 13 and 15 are bonded to the block 9 by means of an adhesive such as Epotek™, or may alternatively be secured by means of pins, staples or other mechanical means. The block 9 extends through the jaw member 4 from the inner surface 7 to the outer surface 8, and has a longitudinal recess 40 in communication with the inner surface, and a longitudinal recess 17 in communication with the outer surface. Located within the recess 40, again secured by a suitable adhesive, is a third electrode 41. Similarly, a fourth electrode 19 is located within the recess 17. The electrodes 41 and 19 are formed of an electrically-conductive metallic material such as stainless steel or tantalum, and extend from the recesses 40 and 17 to project marginally beyond the inner surface 7 and the curved outer surface 8 respectively.

The second jaw member 5 is formed of a rigid substrate material such as steel, plastics, or steel-reinforced plastics material. The second jaw member 5 has an inner surface 21, and this inner surface is covered by a plate 23 formed of an electrically-insulating material such as ceramic, silicone rubber, or a silicone rubber-coated ceramic.

The operation of the instrument of FIGS. 2 to 4 will now be described. When the instrument 12 is to be used to coagulate tissue between the jaw members 4 and 5, the instrument is maneuvered such that the tissue to be coagulated is located between the first and second jaw members 4 and 5. The finger button 6 is operated so as to close the second jaw member 5 against the first jaw member 4, thereby trapping the tissue to be coagulated between the jaw members. As pressure is applied to the tissue by the jaw members 4 and 5, a coagulating voltage is supplied from the generator 10, one pole of the generator being connected to the electrode 13 and the other to electrode 15. Current flows between the electrodes 13 and 15 through the tissue and across the insulating block 9, causing the tissue trapped between the jaw members 4 and 5 to be coagulated.

In an internal cutting mode, the jaw members 4 and 5 remain closed with tissue grasped therebetween. A cutting electrosurgical voltage is supplied from the generator 10, one pole of the generator being connected to the third electrode 41, and the other pole to the electrodes 13 and 15. Arcing occurs between the third electrode 41 and the tissue, and current flows through the tissue to one or both of the electrodes 13 and 15. The cutting of the tissue in this way is more particularly described in our U.S. Pat. No. 7,204,835.

In an external cutting mode, the instrument 12 is maneuvered such that the fourth electrode 19 is in contact with tissue. As before, a cutting electrosurgical voltage is supplied from the generator 10, one pole of the generator being connected to the fourth electrode 19, and the other pole to the electrodes 13 and 15. Arcing occurs between the fourth electrode 19 and the tissue, and current flows through the tissue to whichever of the electrodes 13 and 15 that is also in contact with the tissue. The cutting of the tissue in this way is more particularly described in our U.S. Pat. No. 6,832,998.

The instrument 12 can also be used for spot coagulation, using the fourth electrode 19. In this arrangement, a coagulating voltage is supplied from the generator 10, one pole of the generator being connected to the fourth electrode 19, and the other to the electrodes 13 and 15. Current flows between the fourth electrode 19 through the tissue to the electrodes 13 and 15, causing the tissue in the region of the fourth electrode to be coagulated.

The instrument 12 can also be used in a blended cutting and coagulation mode, in which case an electrosurgical cutting voltage is combined with an electrosurgical coagulating voltage, the cutting voltage being supplied to the third electrode 41 or the fourth electrode 19, and the coagulating voltage to the electrodes 13 and 15. This blended mode of operation is described in our U.S. Pat. No. 6,966,907, and will not be described further herein.

FIG. 5 shows an alternative instrument in which the jaw members 4 and 5 are opened and closed by means of a scissors-type handle arrangement. Referring to FIG. 5, the instrument 12 includes an elongate tubular shaft 27 with a proximal end 28, a distal end 29, and a lumen 30 which extends for the entire length of the tubular member. At the proximal end 28 of the tubular member 27 is a scissors-type handle assembly 31 with a first handle 32 and a second handle 33. The second handle 33 is pivotable with respect to the first handle 32, about pivot pin 34. In a known design of actuation mechanism, the second handle 33 has a pin 35 affixed to its top, such that movement of that handle causes a corresponding movement to a sphere 36 supported in a U-shaped cradle 37.

Fitted into the distal end 29 of the tubular member 27 is a forceps jaw assembly, comprising the first jaw member 4 and the second jaw member 5, pivotally joined to each other by an insulated rivet 38. As described previously, the jaw member 4 is provided with electrodes 13, 15, 19 and 41, these electrodes being supplied by power from the generator 10 by means of leads (not shown) located within the lumen 30 and terminating in a connector 39, by which the instrument 12 can be attached to the generator 10. A push rod 42 also extends through the lumen 30 and acts on links 43 attached to the jaw members 4 and 5. The proximal end of the push rod 42 passes through the sphere 36. In this way, movement of the second handle 33 with respect to the first handle 32 causes a corresponding movement to a sphere 36, the push rod 42, and hence causes the jaw members 4 and 5 to move between their open and closed positions.

FIG. 6 shows an alternative embodiment in which the third and fourth electrodes 41 and 19 are resiliently mounted with respect to the jaw members 4 and 5 respectively. Recesses 17 and 40 not only contain the electrodes 19 and 41, but also blocks 44 and 45 of a resilient material such as rubber or elastomeric polymer. In this way, when the electrode 19 is pressed against tissue, it can move within the recess 17 by compressing the block 44. Similarly, when the jaw members 4 and 5 are closed together grasping tissue therebetween, the electrode 41 can move within the recess 40 by the compression of the block 45. This allows the electrodes 19 and 41 to impose less force on the tissue, such that the tissue is only severed when an electrosurgical cutting voltage is applied to the electrodes, not before.

FIG. 7 shows a slightly different arrangement, in which the electrode 19 only protrudes slightly beyond the inner surface 7 of the jaw member 4, and the electrode 41 only protrudes slightly beyond the outer surface 8 of the jaw member 4. An actuation mechanism (not shown) is provided such that the electrodes 19 and 41 can be mechanically withdrawn into the recesses 17 and 40 such that they are below the inner and outer surfaces 7 and 8 respectively. In this way, the third and fourth electrodes 41 and 19 can be deployed for use, or retracted when not in use. The actuation mechanism can be arranged to advance and retract each electrode 41 and 19, or both electrodes together.

FIG. 8 shows an alternative embodiment in which an electrically-insulating cover 46 is provided to cover the electrode 19 when not required for use. The cover 46 is received within tracks 47 provided in the outer surface 8 of the jaw member 4. The cover 46 is selectively added or removed by the user of the instrument, to either shield or reveal the fourth electrode 19. The cover 46 is either mechanically added and removed by the user, or is deployed and retracted automatically by actuation means (not shown).

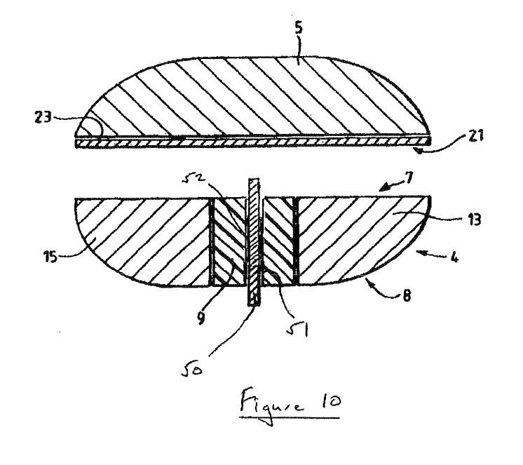

FIG. 9 shows a further embodiment in which the fourth electrode 19 is not opposite the third electrode 41, but is provided on the side face 48 of the first jaw member 4. This enables the instrument 12 to be used in a different way, using the edge of the jaw member 4 to cut or coagulate tissue using the fourth electrode 19.

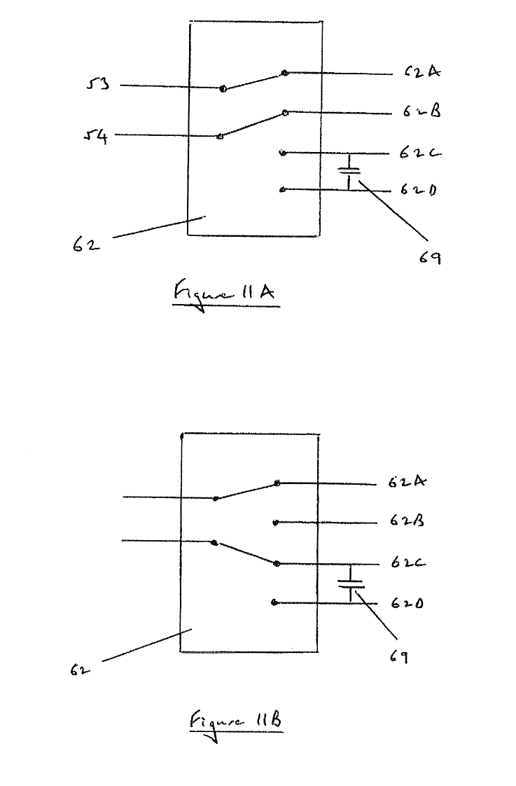

FIG. 10 shows an embodiment in which a single unitary electrode 50 is provided to constitute both the third and fourth electrodes, instead of separate third and fourth electrodes 41 and 19 as in previous embodiments. The unitary electrode 50 is located in a recess 51 running entirely through the central block 9, and is held in place by means of an adhesive, shown at 52. Although this unitary electrode 50 cannot be separately energised as with separate electrode 19 and 41, it does provide a simple and efficient design with considerable robustness and mechanical strength.

FIGS. 11A and 11B show how different electrodes can be connected to the same pole of the generator 10 with a capacitor therebetween. FIG. 11A shows a switching circuit 62 forming part of the output circuit of the generator 10. The RF waveform is supplied to the switching circuit 62 via input connections 53 and 54. The switching circuit 62 has four output connections 62A, 62B, 62C and 62D. Output connections 62A and 62B are connected to the electrodes 13 and 15, and constitute the coagulation output from the generator 10. The output connections 62C and 62D are connected to the electrodes 41 and 19, and constitute the cutting output from the generator 10. A capacitor 69 is present between the output connections 62C and 62D.

FIG. 11A shows the switching circuit in its coagulating position, in which the switching circuit directs the RF waveform between output connections 62A and 62B and hence the coagulation electrodes 13 and 15. The output connections 62C and 62D are isolated, and hence no current flows to the electrodes 41 and 19. FIG. 11B shows the switching circuit in its cutting position, in which the switching circuit directs the RF waveform between output connections 62A and 62C, and hence between the electrodes 13 and 41. As the output connection 62D is also connected to the output connection 62C via the capacitor 69, the electrode 19 is also energised for cutting, albeit at a slightly lower voltage. This allows both cutting electrodes 19 and 41 to be energised from one pole of the electrosurgical generator 10, and also for the cutting action of the electrodes 19 and 41 to be graduated accordingly.

It will be appreciated by those skilled in the art that other combinations of electrode placement and design will be possible without departing from the scope of the invention. For example, the coagulation electrodes can also be connected to the same pole of the generator with a capacitor therebetween. In this way, a differential voltage can be generated between the electrodes without requiring individual output connections for all four (or more) electrodes.

1. A bipolar electrosurgical instrument for cutting and sealing tissue, the instrument comprising:
a) first and second jaw members in opposing relation relative to one another, the first jaw member having an inner surface adapted to cooperate with the inner surface of the second jaw member for grasping tissue therebetween, at least one of the jaw members being movable with respect to the other such that the jaw members are selectively operable between an open position in which the jaw members are disposed in spaced relation relative to one another, and a closed position in which the inner surfaces of the jaw members cooperates for grasping tissue therebetween;
b) means for causing movement of the or each jaw member so as to operate the jaw members between the open and closed positions;
c) a first, coagulating electrode on the inner surface of one of the jaw members;
d) a second, coagulating electrode on the inner surface of one of the jaw members;
e) an insulating member separating the first and second electrodes, the first and second electrodes being connectable to opposite poles of an electrosurgical generator;
f) a third, cutting electrode on the inner surface of the first jaw member, the third electrode being connectable to one pole of the electrosurgical generator; and
g) a fourth electrode on an outer surface of the first jaw member, separate from the inner surface, the fourth electrode being connectable to one pole of the electrosurgical generator;
wherein the electrosurgical instrument is capable of selectively causing coagulation of tissue between the first and second electrodes, and/or cutting of tissue contacted by the third electrode, and/or treatment of tissue contacted by the fourth electrode.

2. An electrosurgical instrument according to claim 1, wherein the fourth electrode is a cutting electrode capable of cutting tissue contacted thereby.

3. An electrosurgical instrument according to claim 1, wherein the fourth electrode is a coagulating electrode capable of coagulating tissue contacted thereby.

4. An electrosurgical instrument according to claim 2, wherein the fourth electrode is an electrode capable of selectively cutting or coagulating tissue contacted thereby.

5. An electrosurgical instrument according to claim 1, wherein the first and second electrodes are provided on the first jaw member.

6. An electrosurgical instrument according to claim 5, wherein the second jaw member has a tissue-contacting surface which is electrically insulating over its entire surface.

7. An electro surgical instrument according to claim 1, wherein the third electrode extends beyond the inner surface of the first jaw member.

8. An electrosurgical instrument according to claim 1, wherein the third electrode is resiliently mounted with respect to the first jaw member.

9. An electrosurgical instrument according to claim 1, wherein the fourth electrode extends beyond the outer surface of the first jaw member.

10. An electrosurgical instrument according to claim 1, wherein the fourth electrode is resiliently mounted with respect to the first jaw member.

11. An electrosurgical instrument according to claim 10, wherein the fourth electrode is retractably mounted with respect to the first jaw member, such that it is movable between a retracted position in which it is below the outer surface of the first jaw member, and an exposed position in which it extends beyond the outer surface of the first jaw member.

12. An electrosurgical instrument according to claim 1, further comprising a removable cover for selectively covering and uncovering the fourth electrode.

13. An electrosurgical instrument according to claim 1, wherein the third and fourth electrodes are separate electrodes.

14. An electrosurgical instrument according to claim 13, wherein the third and fourth electrodes are capable of independent energisation by the electrosurgical generator.

15. An electrosurgical instrument according to claim 13, wherein the third and fourth electrodes are connected one to the other via a capacitor.

16. An electrosurgical generator according to claim 1, wherein the third and fourth electrodes are integrally formed to constitute a single electrode.

17. An electrosurgical system for cutting and sealing tissue, the system comprising an electrosurgical generator and a bipolar electrosurgical instrument, the instrument comprising:
a) first and second jaw members in opposing relation relative to one another, the first jaw member having an inner surface adapted to cooperate with the inner surface of the second jaw member for grasping tissue therebetween, at least one of the jaw members being movable with respect to the other such that the jaw members are selectively operable between an open position in which the jaw members are disposed in spaced relation relative to one another, and a closed position in which the inner surfaces of the jaw members cooperate for grasping tissue therebetween;
b) means for causing movement of the or each jaw member so as to operate the jaw members between the open and closed positions;
c) a first, coagulating electrode on the inner surface of one of the jaw members;
d) a second, coagulating electrode on the inner surface of one of the jaw members;
e) an electrosurgical generator for supplying RF energy to the electrosurgical instrument
f) an insulating member separating the first and second electrodes, the first and second electrodes being connected to opposite poles of the electrosurgical generator;
g) a third, cutting electrode on the inner surface of the first jaw member, the third electrode also being connected to one pole of the electrosurgical generator; and
h) a fourth electrode on an outer surface of the first jaw member, separate from the inner surface, the fourth electrode being connected to one pole of the electrosurgical generator;
the electrosurgical generator being such so to supply RF energy such that the electrosurgical instrument causes coagulation of tissue between the first and second electrodes, and/or cutting of tissue contacted by the third electrode, and/or treatment of tissue contacted by the fourth electrode.

18. An electrosurgical system according to claim 17, wherein the electrosurgical generator supplies cutting RF energy to the fourth electrode such that the electrosurgical instrument causes cutting of tissue contacted by the fourth electrode.

19. An electrosurgical system according to claim 17, wherein the electrosurgical generator supplies coagulating RF energy to the fourth electrode such that the electrosurgical instrument causes coagulation of tissue contacted by the fourth electrode.

20. An electrosurgical system according to claim 18, wherein the electrosurgical generator selectively supplies cutting RF energy and coagulating RF energy to the fourth electrode such that the electrosurgical instrument selectively causes tissue contacted by the fourth electrode to be either cut or coagulated respectively.
